(19) United States
(12) Patent Application Publication
(10) Pub. No.: US 2011/0301676 A1
(43) Pub. Date: Dec. 8, 2011

(54) REDUCING RESONANT CURRENTS IN A RESONATING CIRCUIT DURING MRI SCANS

(75) Inventors: Jin Zhang, Porter Ranch, CA (US); Xiaoyi Min, Thousand Oaks, CA (US); Ingmar Viohl, Milwaukee, WI (US); Gabriel A. Mouchawar, Valencia, CA (US); Xiangqun Chen, Valencia, CA (US)

(73) Assignee: PACESETTER, INC., Sylmar, CA (US)

(21) Appl. No.: 12/792,616

(22) Filed: Jun. 2, 2010

(51) Int. Cl. A61N 1/05; H01R 43/20

(52) U.S. Cl. 607/116; 29/876

(57) ABSTRACT

An implantable medical lead configured to reduce resonant currents in a resonating circuit during MRI scans and a method of manufacturing the same are disclosed herein. The method of manufacturing includes providing a medical lead comprising an electrical pathway from a tip electrode located at a distal end of the lead to a lead connector located at a proximal end and coupling a resonating circuit to the tip electrode such that the resonating circuit is in the electrical pathway for the tip electrode. Further, the method includes coupling a capacitive element to a proximal end of the resonating circuit. The capacitive element is configured to shunt at least part of an RF current induced on the electrical pathway into surrounding tissue or fluid and also works as a heat sink to spread the heat from the internal LC resonant circuit.

### FIELD OF THE INVENTION

The present invention relates to implantable medical leads. More specifically, the present invention relates to implantable medical leads having a lead structure based capacitor to reduce heating.

### BACKGROUND OF THE INVENTION

Existing implantable medical leads for use with implantable pulse generators, such as neurostimulators, pacemakers, defibrillators or implantable cardioverter defibrillators (“ICD”), are prone to heating and induced current when placed in the strong magnetic (static, gradient and RF) fields of a magnetic resonance imaging (“MRI”) machine. The heating and induced current are the result of the lead acting like an antenna in the magnetic fields generated during a MRI. Heating and induced current in the lead may result in deterioration of stimulation thresholds or, in the context of a cardiac lead, even increase the risk of cardiac tissue damage and perforation.

Over fifty percent of patients with an implantable pulse generator and implanted lead require, or can benefit from, a MRI in the diagnosis or treatment of a medical condition. MRI modality allows for flow visualization, characterization of vulnerable plaque, non-invasive angiography, assessment of ischemia and tissue perfusion, and a host of other applications. The diagnosis and treatment options enhanced by MRI are only going to grow over time. For example, MRI has been proposed as a visualization mechanism for lead implantation procedures.

There is a need in the art for an implantable medical lead configured for improved MRI safety. There is also a need in the art for methods of manufacturing and using such a lead.

### BRIEF SUMMARY OF THE INVENTION

In one embodiment of the present invention a capacitive element formed by a portion of a lead structure a method of manufacturing the same for use in an implantable medical lead is disclosed. According to this embodiment of the present invention the capacitive element is configured to shunt RF current induced on a lead conductor into the surrounding tissue or fluid. The capacitive element according to this embodiment is positioned before a resonant component or circuit at or near a distal end of the lead. is disclosed herein with a method of manufacturing the same for use in an implantable medical lead. In accordance with one embodiment, the method of manufacture includes providing a medical lead comprising an electrical pathway from a tip electrode located at a distal end of the lead to a lead connector located at a proximal end and coupling a resonating component or circuit to the tip electrode such that all the resonating component or circuit is in the electrical pathway for the tip electrode. The method also includes coupling a capacitive element to a proximal end of the resonating component or circuit, the capacitive element being configured to shunt at least a portion of an RF current induced on the electrical pathway into the surrounding tissue or fluid.

In another embodiment, a MRI compatible implantable medical lead is provided. The MRI compatible implantable medical lead includes a self-resonant circuit and a tip electrode electrically coupled to a distal end of the self-resonant circuit. The MRI compatible implantable medical lead also includes a capacitive element coupled to the proximal end of the self-resonant circuit.

In yet another embodiment, there is disclosed an implantable medical lead. The implantable medical lead includes a body including a distal portion with an electrode and a proximal portion with a lead connector end and an electrical pathway extending between the electrode and lead connector end, the electrical pathway including an inductor electrically coupled to the electrode. Additionally, the implantable medical lead includes a capacitive element coupled between the inductor and the lead conductor end of the body, the capacitive element being configured to shunt at least a portion of an RF current induced on the lead conductor into the surrounding tissue or fluid.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is an isometric view of an implantable medical lead and a pulse generator for connection thereto.

FIG. 2A is a longitudinal cross-section of a lead distal end having a capacitive element formed in the lead structure body.

FIG. 2B is a simplified schematic diagram of the capacitive element relative to a resonant circuit of the lead distal end of FIG. 2A.

FIG. 3 is a longitudinal cross-section of a lead distal end having multiple capacitive elements formed in the lead structure body.

FIG. 4 is a flowchart illustrating a method of manufacturing an implantable medical lead having a capacitive element formed in the lead body structure for shunting current.

FIG. 5 is a plot showing approximate simulation results for three topologies of implantable medical leads with schematics of the topologies inset into the plot.

FIG. 6 illustrates a plot showing approximate simulation results of the three topologies of implantable medical lead with additional components to simulate an actual medical lead.

### DETAILED DESCRIPTION

Supplying a RF resonant circuit, such as a LC tank circuit, at the distal end of a lead has shown great RF heating reduction independent of lead configuration, lead length and termination conditions. However, recent in-vitro tests also confirmed that due to resonant current at RF frequencies and series resistance, heating of a self-resonant inductor measured at the lead distal end header can be significant in gel (about 19 TC). Component heating in implantable medical leads is due to dissipated power. Power dissipation is represented as P=I²R, where I is electrical resonant current and R is the series resistance at the resonant frequency. Reducing I or R or both can, therefore, reduce heating. Additionally, electrical modeling shows that input current at the resonant circuit is proportional to the resonant current I, therefore, lowering input current at the LC tank is helpful to reduce component heating.

Disclosed herein is an implantable medical lead employing a lead structure based capacitor before a self-resonant inductor in the distal end of the lead for shunting current into surrounding tissue or fluid (or blood). The lead structure based capacitor reduces current flow to the resonating tank circuit resulting in less component heating. The metal surfaces of the capacitor also serve as thermal sinks that spread generated heat and dissipate the heat to the surrounding tissue or fluid.

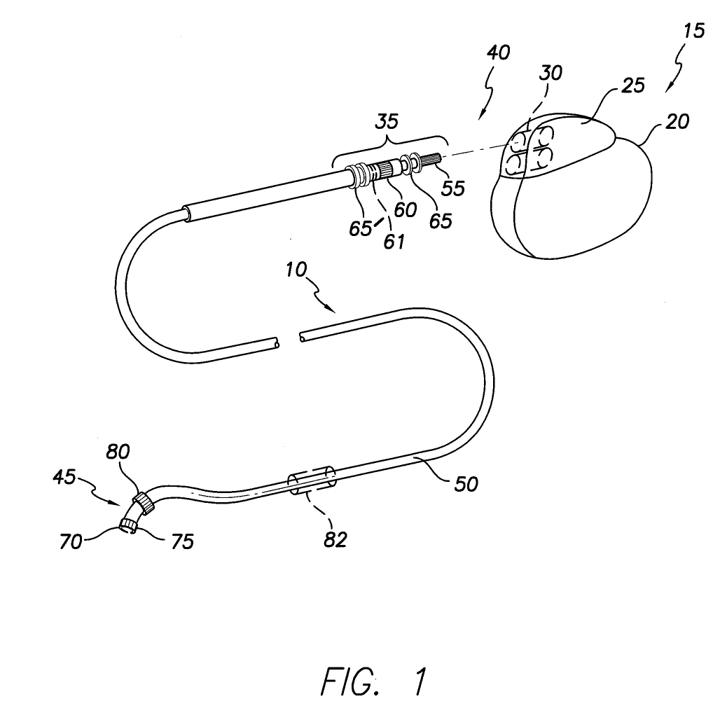

For a general discussion of an embodiment of a lead 10 in which the lead structure based capacitor may be implemented, reference is made to FIG. 1, which is an isometric view of the implantable medical lead 10 and a pulse generator 15 for connection thereto. The pulse generator 15 may be a pacemaker, defibrillator, ICD or neurostimulator. As indicated in FIG. 1, the pulse generator 15 may include a can 20, which may house the electrical components of the pulse generator 15, and a header 25. The header 25 may be mounted on the can 20 and may be configured to receive a lead connector end 35 in a lead receiving receptacle 30. Although only a single lead is illustrated, it can be appreciated that multiple leads may be implemented. In particular, for example, for CRT treatments, there may be leads for both the right and left ventricle.

As shown in FIG. 1, in one embodiment, the lead 10 may include a proximal end 40, a distal end 45 and a tubular body 50 extending between the proximal and distal ends. The lead 10 may be configured for a variety of uses. For example, the lead 10 may be a RA lead, RV lead, LV Brady lead, RV Tachy lead, intrapericardial lead, etc.

As indicated in FIG. 1, the proximal end 40 may include a lead connector end 35 including a pin contact 55, a first ring contact 60, a second ring contact 61, which is optional, and sets of spaced-apart radially projecting seals 65. In some embodiments, the lead connector end 35 may include the same or different seals and may include a greater or lesser number of contacts. The lead connector end 35 may be received in a lead receiving receptacle 30 of the pulse generator 15 such that the seals 65 prevent the ingress of bodily fluids into the respective receptacle 30 and the contacts 55, 60, 61 electrically contact corresponding electrical terminals within the respective receptacle 30.

As illustrated in FIG. 1, in one embodiment, the lead distal end 45 may include a distal tip 70, a tip electrode 75 and a ring electrode 80. In some embodiments, the lead body 50 is configured to facilitate passive fixation and/or the lead distal end 45 includes features that facilitate passive fixation. In such embodiments, the tip electrode 75 may be in the form of a ring or domed cap and may form the distal tip 70 of the lead body 50.

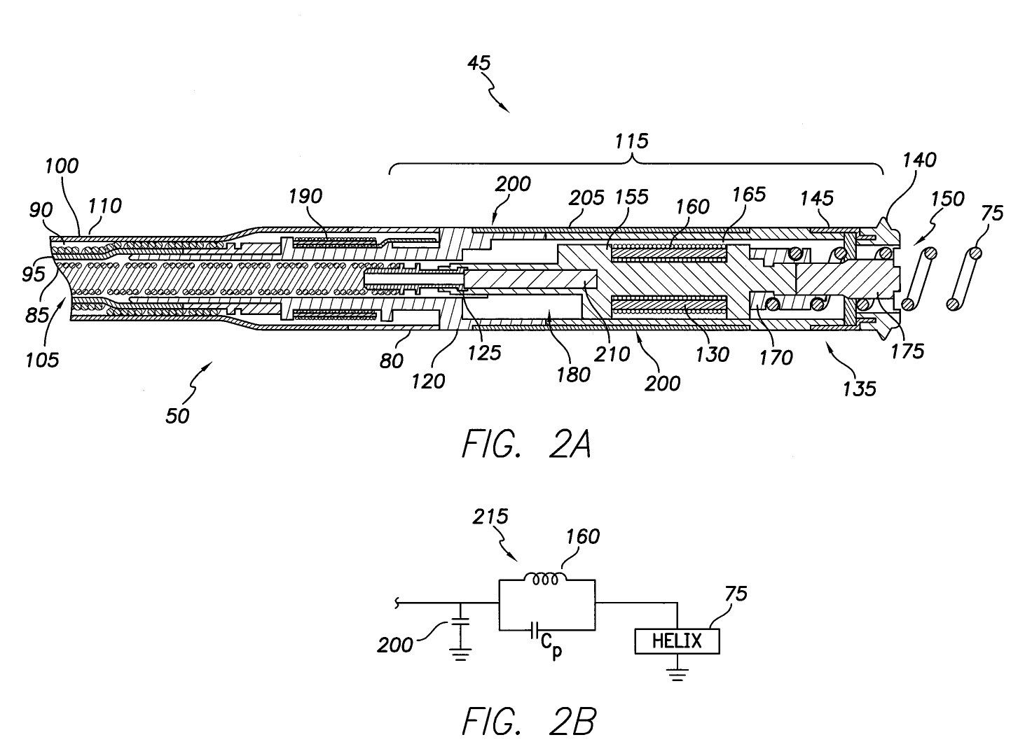

As shown in FIG. 2A, which is a longitudinal cross-section of the lead distal end 45, in some embodiments, the tip electrode 75 may be in the form of a helical anchor 75 that is extendable from within the distal tip 70 for active fixation and serving as a tip electrode 75.

As shown in FIG. 1, in some embodiments, the distal end 45 may include a defibrillation coil 82 about the outer circumference of the lead body 50. The defibrillation coil 82 may be located proximal of the ring electrode 70.

The ring electrode 80 may extend about the outer circumference of the lead body 50, proximal of the distal tip 70. In other embodiments, the distal end 45 may include a greater or lesser number of electrodes 75, 80 in different or similar configurations.

As can be understood from FIGS. 1 and 2A, in one embodiment, the tip electrode 75 may be in electrical communication with the pin contact 55 via a first electrical conductor 85, and the ring electrode 80 may be in electrical communication with the first ring contact 60 via a second electrical conductor 90. In some embodiments, the defibrillation coil 82 may be in electrical communication with the second ring contact 61 via a third electrical conductor. In yet other embodiments, other lead components (e.g., additional ring electrodes, various types of sensors, etc.) (not shown) mounted on the lead body distal region 45 or other locations on the lead body 50 may be in electrical communication with a third ring contact (not shown) similar to the second ring contact 61 via a fourth electrical conductor (not shown). Depending on the embodiment, any one or more of the conductors 85, 90 may be a multi-strand or multi-filar cable or a single solid wire conductor run singly or grouped, for example in a pair.

As shown in FIG. 2A, in one embodiment, the lead body 50 proximal of the ring electrode 80 may have a concentric layer configuration and may be formed at least in part by inner and outer helical coil conductors 85, 90, an inner tubing 95, and an outer tubing 100. The helical coil conductor 85, 90, the inner tubing 95 and the outer tubing 100 form concentric layers of the lead body 50. The inner helical coil conductor 85 forms the inner most layer of the lead body 50 and defines a central lumen 105 for receiving a stylet or guidewire therethrough. The inner helical coil conductor 85 is surrounded by the inner tubing 95 and forms the second most inner layer of the lead body 50. The outer helical coil conductor 90 surrounds the inner tubing 95 and forms the third most inner layer of the lead body 50. The outer tubing 100 surrounds the outer helical coil conductor 90 and forms the outer most layer of the lead body 50.

In one embodiment, the inner tubing 95 may be formed of an electrical insulation material such as, for example, ethylene tetrafluoroethylene (“ETFE”), polytetrafluoroethylene (“PTFE”), silicone rubber, silicone rubber polyurethane copolymer (“SPC”), or etc. The inner tubing 95 may serve to electrically isolate the inner conductor 85 from the outer conductor 90. The outer tubing 100 may be formed of a biocompatible electrical insulation material such as, for example, silicone rubber, silicone rubber-polyurethane-copolymer (“SPC”), polyurethane, gore, or etc. The outer tubing 100 may serve as the jacket 100 of the lead body 50, defining the outer circumferential surface 110 of the lead body 50.

As illustrated in FIG. 2A, in one embodiment, the lead body 50 in the vicinity of the ring electrode 80 transitions from the above-described concentric layer configuration to a header assembly 115. For example, in one embodiment, the outer tubing 100 terminates at a proximal edge of the ring electrode 80, the outer conductor 90 mechanically and electrically couples to a proximal end of the ring electrode 80, the inner tubing 95 is sandwiched between the interior of the ring electrode 80 and an exterior of a proximal end portion of a body 120 of the header assembly 115, and the inner conductor 85 extends distally past the ring electrode 80 to electrically and mechanically couple to components of the header assembly 115 as discussed below.

As depicted in FIG. 2A, in one embodiment, the header assembly 115 may include the body 120, a coupler 125, an inductor assembly 130, and a helix assembly 135. The header body 120 may be a tube forming the outer circumferential surface of the header assembly 115 and enclosing the components of the assembly 115. The header body 120 may have a soft atraumatic distal tip 140 with a radiopaque marker 145 to facilitate the soft atraumatic distal tip 140 being visualized during fluoroscopy. The distal tip 140 may form the extreme distal end 70 of the lead 10 and includes a distal opening 150 through which the helical tip anchor 75 may be extended or retracted. The header body 120 may be formed of polyetheretherketone (“PEEK”), polyurethane, or etc., the soft distal tip 140 may be formed of silicone rubber, SPC, or etc., and the radiopaque marker 145 may be formed of platinum, platinum-iridium alloy, tungsten, tantalum, or etc.

As indicated in FIG. 2A, in one embodiment, the inductor assembly 130 may include a bobbin 155, an inductor 160 and a shrink tube 165. The bobbin 155 may include a proximal portion that receives the coupler 125 such that the coupler 125 and bobbin 155 are mechanically coupled to each other. The bobbin 155 may also include a barrel portion about which the inductor 160 is located and a distal portion coupled to the helix assembly 135. The bobbin 155 may be formed of an electrical insulation material such as PEEK, polyurethane, or etc.

To achieve a self resonant frequency (SRF) close to 64 MHz or 128 MHz and have sufficiently high impedance (usually greater than 1000Ω), the inductor 160 is wound with many turns in multiple layers. The insulated wire of the inductor 160 and the multilayered tight winding can generate strong mutual inductance and parasitic capacitance between the tight coil turns and coil layers. Hence, the inductor 160 may function as a LC tank resonant circuit that commonly is referred to as a “tank filter.” Generally, energy oscillates back and forth between the capacitive element and the inductor 160. This resonant circuit will create a high impedance close to the resonant frequency.

As illustrated in FIG. 2A, the shrink tube 165 may extend about the inductor 160 to generally enclose the inductor 160 within the boundaries of the bobbin 155 and the shrink tube 165. The shrink tube 165 may act as a barrier between the inductor 160 and the inner circumferential surface of the header body 120. Also, the shrink tube 165 may be used to form at least part of a hermitic seal about the inductor 160. The shrink tube 165 may be formed of fluorinated ethylene propylene (“FEP”), polyester, or etc.

As shown in FIG. 2A, a distal portion of the coupler 125 may be received in the proximal portion of the bobbin 155 such that the coupler 125 and bobbin 155 are mechanically coupled to each other. A proximal portion of the coupler 125 may be received in the lumen 105 of the inner coil conductor 85 at the extreme distal end of the inner coil conductor 85, the inner coil conductor 85 and the coupler 125 being mechanically and electrically coupled to each other. The coupler 125 may be formed of MP35N, platinum, platinum iridium alloy, stainless steel, etc.

As indicated in FIG. 2A, the helix assembly 135 may include a base 170, the helical anchor electrode 75, and a steroid plug 175. The base 170 forms the proximal portion of the assembly 135. The helical anchor electrode 75 forms the distal portion of the assembly 135. The steroid plug 175 may be located within the volume defined by the helical coils of the helical anchor electrode 75. The base 170 and the helical anchor electrode 75 are mechanically and electrically coupled together. The distal portion of the bobbin 155 may be received in the helix base 170 such that the bobbin 155 and the helix base 170 are mechanically coupled to each other. The base 170 of the helix assembly 135 may be formed of platinum, platinum-iridium alloy, MP35N, stainless steel, or etc. The helical anchor electrode 75 may be formed of platinum, platinum-iridium ally, MP35N, stainless steel, or etc.

As can be understood from FIG. 2A and the preceding discussion, the coupler 125, inductor assembly 130, and helix assembly 135 are mechanically coupled together such that these elements 125, 130, 135 of the header assembly 115 do not displace relative to each other. Instead these elements 125, 130, 135 of the header assembly 115 are capable of displacing as a unit relative to, and within, the body 120 via the pin contact 55, which is rotatable relative to the rest of the lead connector end 35 and is mechanically and electrically coupled to the proximal end of the inner coil 85, the inner coil 85 being rotatable relative to the rest of the lead body 50. In other words, these elements 125, 130, 135 of the header assembly 115 form an electrode-inductor assembly 180, which can be caused to displace relative to, and within, the header assembly body 120 when a pin contact 55 and the inner coil 85 are caused to rotate within the lead connector end 35 and the lead body 50, respectively. Specifically, the pin contact 55 is rotated relative to the lead connector end 35, which causes the inner coil 85 to rotate relative to the lead body 50, which in turn causes the electrode-inductor assembly 180 to rotate within the header assembly of the lead distal end. Thus, rotation of the electrode-inductor assembly 180 in a first direction via rotation of the pin contact 55 in the first direction causes the electrode-inductor assembly 180 to displace distally, and rotation of the electrode-inductor assembly 180 in a second direction opposite the first direction via rotation of the pin contact 55 in the second direction causes the electrode-inductor assembly 180 to retract into the header assembly body 120. Thus, causing the electrode-inductor assembly 180 to rotate within the body 120 in a first direction causes the helical anchor electrode 75 to emanate from the tip opening 150 for screwing into tissue at the implant site. Conversely, causing the electrode-inductor assembly 180 to rotate within the body 120 in a second direction causes the helical anchor electrode 75 to retract into the tip opening 150 to unscrew the anchor 75 from the tissue at the implant site.

As already mentioned and indicated in FIG. 2A, the inductor 160 may be positioned about the barrel portion of the bobbin 155. A proximal end of the inductor 160 may extend through the proximal portion of the bobbin 155 to electrically couple with the coupler 125, and a distal end of the inductor 160 may extend through the distal portion of the bobbin 155 to electrically couple to the helix base 170. Thus, in one embodiment, the inductor 160 is in electrical communication with the both the inner coil conductor 85, via the coupler 125, and the helical anchor electrode 75, via the helix base 170. Therefore, the inductor 160 acts as an electrical pathway through the electrically insulating bobbin 155 between the coupler 125 and the helix base 170. In one embodiment, all electricity destined for the helical anchor electrode 75 from the inner coil conductor 85 passes through the inductor 160 such that the inner coil conductor 85 and the electrode 75 both benefit from the presence of the inductor 160, the inductor 160 acting as a self resonant LC tank circuit 160 when the lead 10 is present in a magnetic field of a MRI. The capacitor is made by the parasitic capacitance between coil to coil of the inductor.

In one embodiment, the inductor is a LC tank circuit or a self-resonant inductor. In one version of such an embodiment, the capacitor does not exist in the LC tank circuit, instead relying on the parasitic capacitance that exists between each coil of the inductor. As a result, a whole inductor becomes a LC tank resonant circuit. The implement of such a self-resonant inductor takes care of both the inductor value and the parasitic capacitance value.

As the helix base 170 may be formed of a mass of metal, the helix base 170 may serve as a relatively large heat sink for the inductor 160, which is physically connected to the helix base 170. Similarly, as the coupler 125 may be formed of a mass of metal, the coupler 125 may serve as a relatively large heat sink for the inductor 160, which is physically connected to the coupler 125.

Some lead embodiments may have both a tip inductor 160 and a ring inductor 190. In such embodiments, the ring inductor 190 is part of the electrical circuit extending between the ring electrode 80 and the outer conductor 90 and the tip inductor 160 is part of the electrical circuit between the tip electrode 75 and the inner conductor 85. In such an embodiment, decoupling or isolating of the tip inductor 160 from ring inductor 190 may be implemented as one or more magnetic shielding layers (“shield”) or a non-magnetic, electrically conductive material. In other embodiments, shields may not be located between the inductors 160, 190 and the two inductors 160, 190 may not be magnetically decoupled.

Additionally, in some embodiments, the tip inductor 160 may have a self-resonant frequency (SRF) that is different from the SRF of the ring inductor 190. For example, one of the inductors 160, 190 may be tuned for a frequency of 64 MHz and the other of the inductors may be tuned for a frequency of 128 MHz. Alternatively, in some embodiments, the tip inductor 160 may have a SRF that is the same as the SRF of the ring inductor 190. For example, both of the inductors 160, 190 may be tuned for a frequency of 64 MHz or 128 MHz.

As illustrated, in FIG. 2A, the lead structure includes a capacitive element 200. That is the capacitive element 200 is integral to the lead body structure. The capacitive element 200 includes a first metallic member 205 and a second metallic member 210. The first and second metallic members 205 and 210 may be made of any suitable non-magnetic material that is both electrically and thermally conductive, such as, for example, titanium, platinum, platinum iridium alloy, etc. The first metallic member 205 may be a floating metal cylinder located on an exterior surface of the lead structure. In other embodiments, the first metallic member 205 may be a metallic strip, a metallic semicircular member, or other suitable geometric shape located on the exterior surface of the lead. As the first metallic member 205 is floating, it is not electrically coupled to wires, filars, cables or other electrical conductors or to other electrical components of the lead. The surface of the first metallic member 205 provides thermal spreading and/or heat sinking to cool the inductor body, as shown in FIG. 2A. Thus, the inductor body 160 can act as one node of the capacitor (faces the external blood flow) and also act as a heat sink to spread the heat generated from the inductor body (the LC tank resonant circuit 160).

The second metallic member 210 may be concentrically located within the lead structure and the first metallic member 205. In one embodiment, the second metallic member 210 forms the electrical connection between the inner coil 85 and the tip inductor 160. The second metallic member 210 may be a solid member or a hollow member and may have any suitable geometric shape, such as cylinder, rod, etc.

The capacitive effects of the capacitive element 200 may be influenced by a number of factors, such as common surface area of the members 205 and 210, the distance separating the plates, the permittivity of the material separating the members 205 and 210 and the RF frequency, for example. Generally, the shunting capacitance of the capacitive element 200 may be 30 pF or greater. In one embodiment, the shunting capacitance may be between 60 and 100 pF, such as 80 pF, for example.

In one embodiment, the shunting capacitance may be 30 pF. The shunting capacitance may depend on the surface area of features 210 and 205, and will generally increase with increasing surface areas of features 205 and 210. The insulation material may be the same as the dielectric material in a real capacitor. Shunting capacitance may also vary depending on the effective distance between 205 and 210 for the specific embodiment. In some cases, the blood flow between features 210 and 205 may be considered as another layer of dielectric material.

The capacitive element 200 directs the shunted part of the current into surrounding tissue or fluid and reduces resonant current within the tip inductor (LC tank) 160 to reduce component heating. Specifically, the capacitive element 200 shunts at least a portion of the current at RF frequency into the surrounding tissue or fluid before it enters into a self resonant inductor or LC tank resonant circuit, such as the inductor 160. FIG. 2B illustrates a simplified schematic of the electrical components of the lead. Specifically, FIG. 2B shows the capacitor 200 in an electrically parallel configuration relative to the helix electrode 75. Additionally, the capacitor 200 is located in the circuit before the self-resonant circuit 160. That is, the capacitor 200 is on the proximal end of the inductor 160.

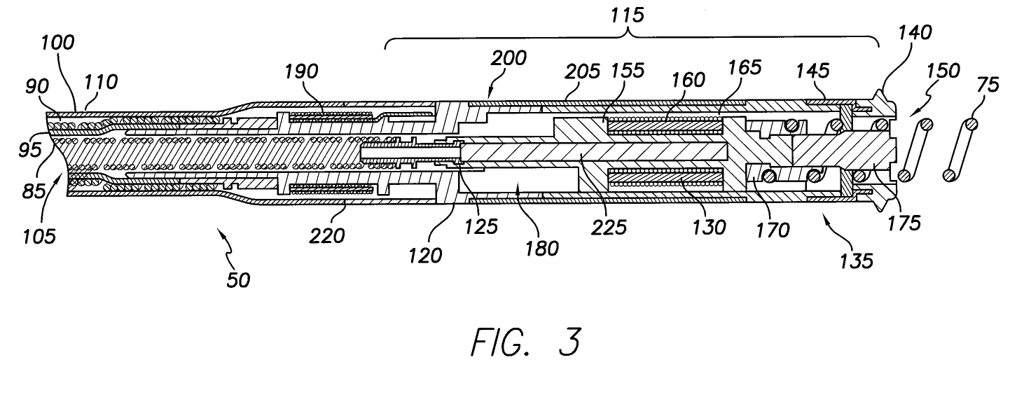

FIG. 3 illustrates an alternative embodiment wherein a ring electrode 220 is configured as a thermal sink that may offer some capacitance in some embodiments, and a second metallic member 225 of the tip capacitor 200 is elongated. In other aspects the lead shown in FIG. 3 is similar to the lead illustrated in FIG. 2A.

As can be seen in FIG. 3, in this embodiment, the ring electrode 220 is configured as a thermal sink. As such, the ring electrode 220 may be referred to as the ring thermal sink 220. The ring 220 is positioned on the exterior of the lead about the ring inductor 190. Although modified to function as a thermal sink, the ring 220 remains electrically coupled to the ring inductor 190 so that it may continue to also function as the ring electrode. Although the ring 220 is configured primarily as a thermal sink to spread the heat from the self resonant circuit 190, the ring may provide some capacitance, although not a dominate capacitor effect.

FIG. 3 also shows a modified second metallic member 225 of the tip capacitor 200. As can be seen, the second metallic member 225 of the tip capacitor 200 is elongated to extend from the coupler 125 through the center of the inductor 160. As such, in this embodiment the second metallic member 225 forms the core of the tip inductor 160.

In addition to shunting capacitance provided by the capacitive elements 200 and 220, the capacitive elements 200 and 220 can enhance frequency characteristics of the filters. Specifically, by choosing appropriate designs of the inductor and capacitor, the bandwidth may be broadened such that sufficient impedance may be provided for RF frequencies of both 1.5T MRI and 3.0T MRI machines which operate at 64 MHz and 128 MHz respectively.

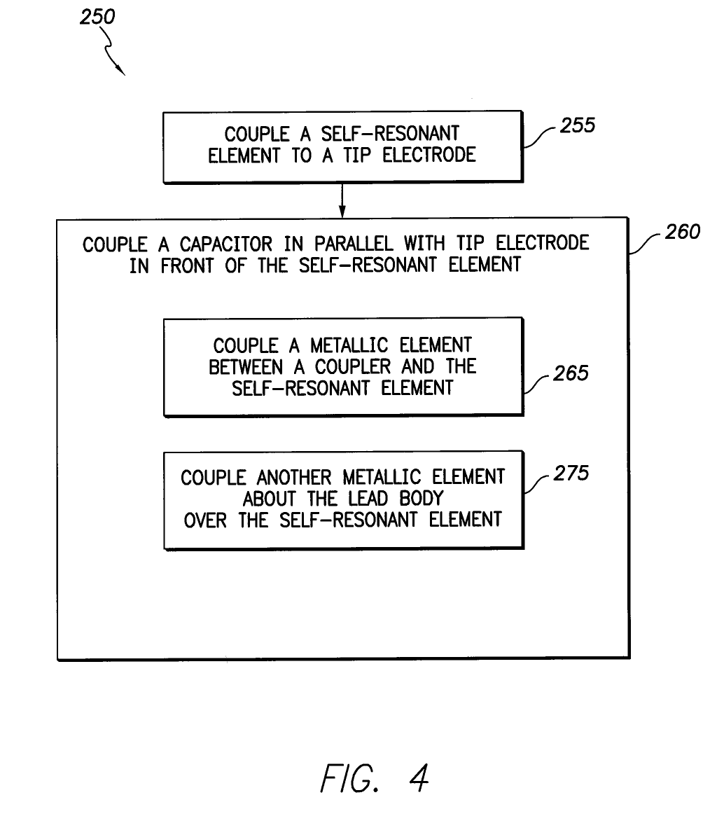

FIG. 4 is a flowchart illustrating a method 250 of manufacturing a MRI compatible implantable lead with a capacitive element to reduce component heating in accordance with an embodiment. The method 250 includes coupling a self-resonant element to a tip electrode, as indicated at block 255. The self-resonant element is electrically coupled to both the tip electrode and a coupler, such that the self-resonant element is the electrical pathway between the coupler and the tip electrode. Referring to FIG. 3, the tip inductor 160 is an example of a self-resonant element and the helix 75 is an example of a tip electrode. It should be understood that other embodiments may include other components or configurations. For example, a self-resonant circuit that includes both an inductor and a capacitor may be implemented rather than the self-resonant inductor 160.

The method 250 illustrated in FIG. 4 also includes coupling a capacitor in an electrically parallel configuration with the tip electrode and in front of the self-resonant element, as indicated at block 260. The coupling of the capacitor may include coupling a metallic element between the coupler and the self-resonant element, as indicated at block 265. The metallic element may take various forms, as discussed above. Specifically, in the embodiment illustrated in FIG. 2A, the metallic element 210 only extends to a bobbin 155 about which the inductive coils or inductor 160 are wound. In another embodiment illustrated in FIG. 3, the metallic element 225 extends through the bobbin 155 and may serve as the core for inductor 160.

The coupling of the capacitor in an electrically parallel configuration with the tip electrode and in front of the self-resonant element also includes providing another metallic element about the lead body over the self-resonant element, as indicated at block 275. The other metallic element is externally coupled to the lead body such that it may be in contact with fluid or tissue when in use. The other metallic element is not otherwise electrically coupled to other component parts of the lead and, as such, may be referred to as “floating.” However, the other metallic element provides capacitive effects in conjunction with the metallic element described in block 270. In FIGS. 2A and 3, the other metallic element is represented as element 205.

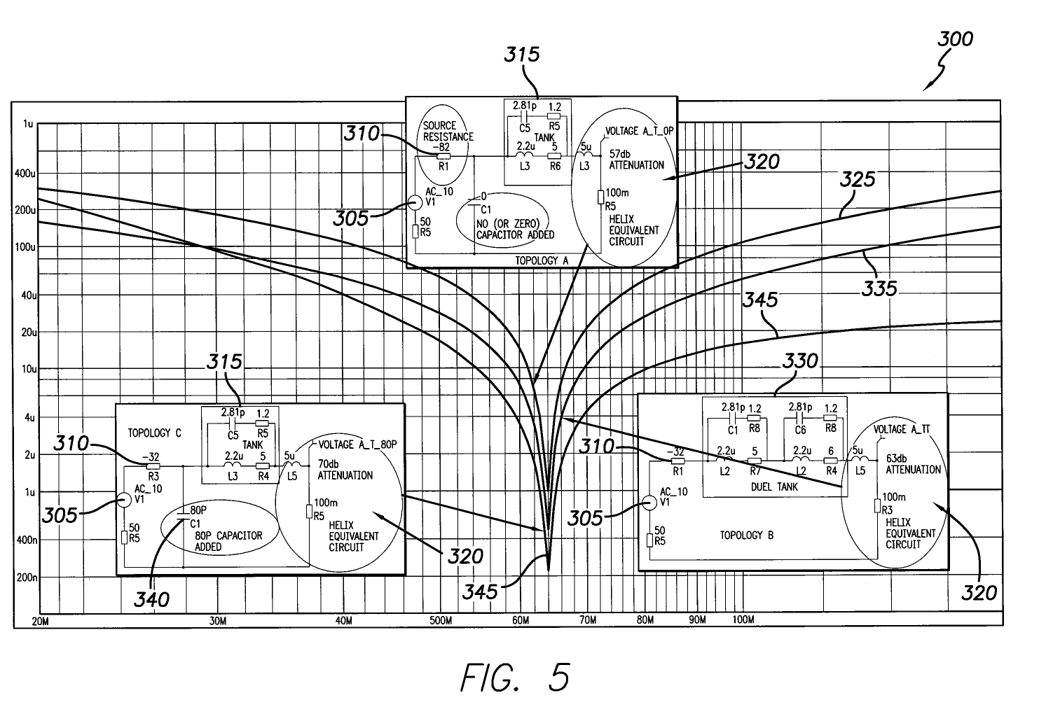

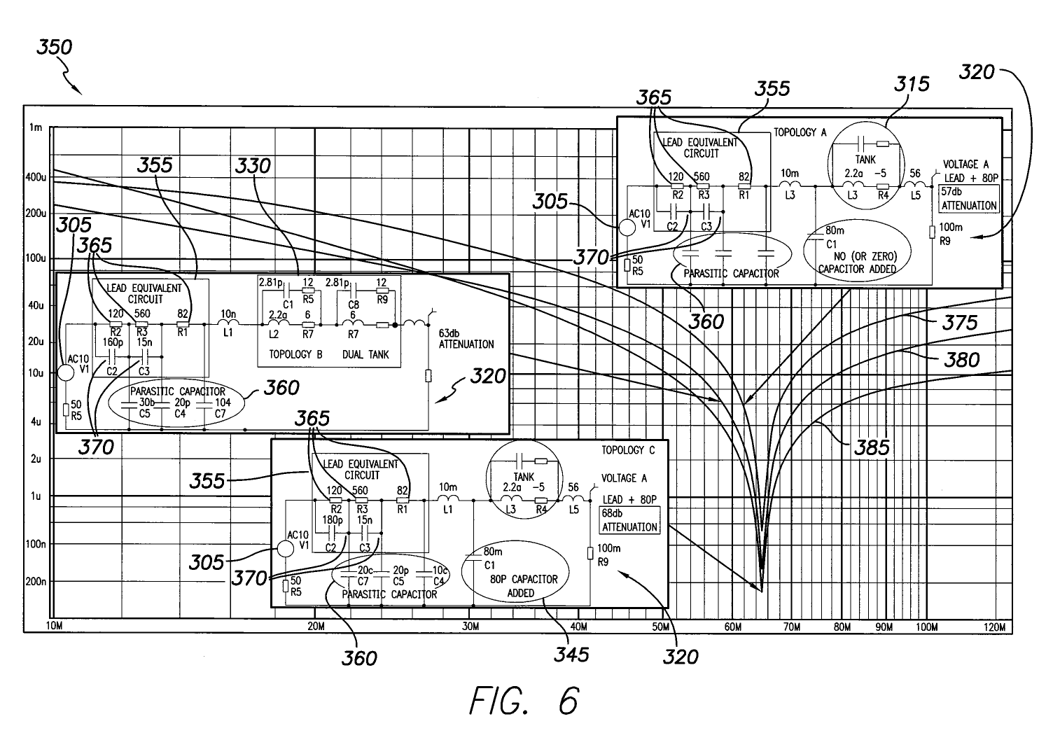

FIGS. 5 and 6 illustrate simplified simulation results comparing three different filter topologies for MRI compatible leads. In the simulations, 3D electromagnetic models were used and a compartment in the lead header was filled with fluid to simulate a chronic condition. As discussed above, when the lead is installed for use, the tip electrode 75 and the capacitor 200 are coupled in an electrically parallel configuration, as both the tip electrode 75 and the capacitor 200 are coupled to fluid or tissue.

Turning to FIG. 5, a plot 300 of approximate simulation results for three different topologies is illustrated. Each of the different topologies are illustrated as schematic insets in the plot 300. The horizontal axis of the plot 300 represents frequency in Megahertz and the vertical axis represents attenuation in voltage. Topology A is illustrated in the inset schematic located near the top of the plot 300. As can be seen Topology A includes an AC source 305 with source resistance 310, a tank filter 315, and a helix equivalent circuit 320. However, no capacitor is provided in parallel with the helix equivalent circuit 320 before the tank filter 315. Topology A achieved 57 db of attenuation in the simulation, as represented by plot line 325 in the plot 300.

Topology B is illustrated in the lower right-hand side of the plot 300 and includes the AC source 305 with source resistance 310. A dual tank filter 330 is provided in an electrically serial configuration with the AC source 305 and the helix equivalent circuit 320. Similar to Topology A, no capacitor is provided in parallel with the helix equivalent circuit 320. Topology B with the dual tank filters 330 achieved 63 db of attenuation, as represented by plot line 335 in plot 300.

Topology C is illustrated in the lower left-hand side of the plot 300 and includes the AC source 305 with series resistance 310, the single tank filter 315, and the helix equivalent circuit 320. In contrast to Topologies A and B, Topology C includes an 80 pF capacitive element 340 in parallel with the helix equivalent circuit 320. The capacitive element 345 is located between the AC source 305 and the tank filter 315 to provide shunting capacitance. That is, the capacitive element is located before the tank filter 315. In the simulation, Topology C achieved an attenuation of 70 db, as represented by plot line 345. Hence, the addition of an 80 pF capacitor before the tank filter greatly increased the attenuation level even beyond the attenuation achieved with a dual tank filter as in Topology B.

Turning to FIG. 6, a plot 350 illustrates the results of a simulation of Topologies A, B and C with an additional lead equivalent circuit 355 and distributive parasitic capacitors 360. As can be seen in FIG. 6, the lead equivalent circuit 355 includes resistors 365 and capacitors 370, as well as parasitic capacitors 360, to imitate the resistance and capacitance that would be present in an actual lead. Again, each of the topologies are illustrated as inset schematic diagrams in the plot 350. The schematic of Topology A is located near the top of the plot 350, the schematic of Topology B is illustrated on the left-hand side of the plot 350 and the schematic of Topology C is illustrated near the bottom of the plot 350.

As in the simulation of FIG. 5, Topology C includes the 80 pF capacitive element 345 located before the single tank filter 315. Topology A and B do not include the capacitive element 345. Rather, they simply include the single tank filter 315 and dual tank filter 330, respectively. As shown in FIG. 6, Topology A achieved 57 db attenuation, as indicated by plot line 375, Topology B achieved 63 db attenuation, as indicated by plot line 380, and Topology C achieved 68 db attenuation, as indicated by plot line 385. Hence, even a more realistic simulation including the lead equivalent circuit 355 and parasitic capacitors 360, Topology C demonstrated better attenuation with the capacitive element 345 than Topologies A and B which do not include the capacitive element 345.

The foregoing describes some example embodiments to reduce component heating during MRI scans by reducing resonant current inside a resonating circuit. The reduction in resonant current is achieved by shunting current into fluid or tissue. In particular, a capacitive element is provided in front of the resonant circuit. The capacitive element directs shunting current into fluid or tissue. This reduces resonant current in the resonant circuit resulting in less component heating. The capacitive element includes a metallic element that is externally exposed from the lead body such that it may be in contact with the fluid or tissue. In addition to reducing the heat in the resonating circuit, the metal surfaces of the capacitive element also serve as thermal spreaders and/or heat sinks that benefit cooling. Although the present invention has been described with reference to illustrated embodiments, persons skilled in the art will recognize that changes may be made in form and detail without departing from the spirit and scope of the invention. Indeed, in other embodiments, one or more additional capacitive elements may be coupled to the lead. Additionally, capacitive elements may be implemented with different filtering techniques. For example, although not described herein, a capacitive element may be used in conjunction with a dual tank filter or other filter. Accordingly, the specific embodiments described herein should be understood as examples and not limiting the scope of the disclosure.

What is claimed is:

1. A method of manufacturing a MRI compatible implantable medical lead comprising:
providing a medical lead comprising an electrical pathway from a tip electrode located at a distal end of the lead to a lead connector located at a proximal end;
coupling a resonating circuit to the tip electrode such that the resonating circuit is in the electrical pathway for the tip electrode; and
coupling a capacitive element to a proximal end of the resonating circuit, the capacitive element being configured to shunt at least part of an RF current induced on the electrical pathway into surrounding tissue or fluid.

2. The method of manufacturing of claim 1 wherein coupling a capacitive element to the resonating circuit comprises:
coupling a metallic member between a coupler and the resonating circuit; and
coupling a floating metallic member about a lead body over the resonating circuit.

3. The method of manufacture of claim 2 comprising providing a ring electrode that extends around the body of the lead and has a length greater than a ring inductor.

4. The method of manufacturing of claim 2 wherein coupling the metallic member in between the coupler and the resonating circuit comprises coupling a metallic member between the coupler and a bobbin about which a component of the resonating circuit is wound.

5. The method of manufacturing of claim 2 wherein coupling the metallic member in between the coupler and the resonating circuit comprises coupling a metallic member between the coupler and through the center of a bobbin about which a component of the resonating circuit extends, such that the metallic member is the core of the resonating circuit.

6. The method of manufacturing of claim 2 wherein coupling the floating metallic member about the lead body comprises exposing the floating metallic member externally from the lead body.

7. The method of manufacturing of claim 1 comprising configuring the capacitive element to provide greater than 30 pF of capacitance.

8. The method of manufacturing of claim 1 comprising configuring the resonating circuit to have a self-resonant frequency of 64 MHz or 128 MHz.

9. The method of manufacturing of claim 1 further comprising configuring a metallic side of the capacitive element to operate as both a heat sink to spread the heat generated from the resonant circuit and a current shunt.

10. A MRI compatible implantable medical lead comprising:
a lead body having an electrical conductor adapted to couple a connector to a tip electrode;
a self-resonant circuit electrically coupled between a distal end of the electrical conductor and the tip electrode; and
a capacitive element formed in the body of the lead and coupled to the proximal end of the self-resonant circuit, wherein the capacitive element is adapted to shunt at least a portion of an RF current induced on the electrical conductor into surrounding tissue or fluid.

11. The MRI compatible implantable medical lead of claim 10 wherein the capacitive element comprises:
a first metallic member electrically coupled in between a coupler and the self-resonant circuit; and
a second metallic member mechanically coupled about the body of the lead over the self-resonating circuit.

12. The MRI compatible implantable medical lead of claim 11 wherein the second metallic member comprises a cylindrical shape and is externally exposed from the lead body such that when the MRI compatible implantable medical lead is in use the second metallic member is in contact with fluid or tissue.

13. The MRI compatible implantable medical lead of claim 12 wherein the second metallic member is configured to operate as both a heat sink to spread the heat generated from the self-resonant circuit and a current shunt.

14. The MRI compatible implantable medical lead of claim 10 wherein the capacitive element is configured to provide 30 pF or greater capacitance.

15. The MRI compatible implantable medical lead of claim 10 wherein the self-resonating circuit resonates at 64 MHz or 128 MHz.

16. The MRI compatible implantable medical lead of claim 10 wherein the self-resonant circuit comprises an inductor and a capacitor.

17. The MRI compatible implantable medical lead of claim 10, wherein the lead is an active fixation lead or a passive fixation lead.

18. An implantable medical lead comprising:
a body including a distal portion with an electrode and a proximal portion with a lead connector end; and
an electrical conductor extending between the electrode and lead connector end,
an inductor electrically coupled in series in the electrical pathway between a distal end of the electrical conductor and the electrode; and
a capacitive element coupled between inductor and the distal end of the electrical conductor, the capacitive element being configured to shunt at least part of an RF current induced on the electrical conductor into surrounding tissue or fluid.

19. The implantable medical lead of claim 18, wherein the capacitive element is further configured to transfer heat.

20. The implantable medical lead of claim 18 wherein the capacitive element comprises:
a metallic member electrically coupled to the inductor; and
a floating metallic member externally exposed from the body and coupled about the body over the inductor, the floating metallic member configured to be both electrically and thermally coupled in parallel with the electrode when the lead is implanted.

21. The implantable medical lead of claim 19 wherein the second metallic member is mechanically coupled to a bobbin about which the inductor is wound.

22. The implantable medical lead of claim 19 wherein the second metallic member extends through a bobbin about which the inductor is wound.

23. The implantable medical lead of claim 22 wherein the second metallic member helps to transfer and spread part of the RF heat generated from the inductor.

24. The implantable medical lead of claim 18 further comprising a ring capacitive element comprising an electrode and thermally coupled about the body over a ring inductor.
